(19) United States
(12) Patent Application Publication
(10) Pub. No.: US 2021/0134464 A1
(43) Pub. Date: May 6, 2021

(54) HEALTH CONDITION INSPECTION SYSTEM

(71) Applicant: Japan Display Inc., Tokyo (JP)

(72) Inventors: Taiki KASAI, Tokyo (JP); Makoto WATANABE, Tokyo (JP)

(73) Assignee: Japan Display Inc., Tokyo (JP)

(21) Appl. No.: 17/149,991

(22) Filed: Jan. 15, 2021

Related U.S. Application Data
(63) Continuation of application No. PCT/JP2019/027824, filed on Jul. 12, 2019.

(30) Foreign Application Priority Data
Jul. 30, 2018 (JP) 2018-142559

(51) Int. Cl. G16H 50/20; G16H 10/60; G16H 40/67; G06N 20/00; A61B 10/00; A61B 5/1172; G06K 9/00; G06K 9/62; G01N 21/3563; G01N 33/483

(52) U.S. Cl. CPC G16H 50/20; G16H 10/60; G16H 40/67; G06N 20/00; A61B 10/0038; A61B 5/1172; G01N 33/4833; G06K 9/00013; G06K 9/00926; G06K 9/00892; G06K 9/6267; G01N 21/3563; G06K 9/00087

(57) ABSTRACT

According to one embodiment, a health condition inspection system includes a first light source, first infrared light sensors, and a processor. The first light source irradiates infrared light to a fecal matter of a subject, discharged into a toilet. The first infrared light sensors are arranged inside the toilet and configured to receive the infrared light irradiated from the first light source. The processor is configured to inspect a health condition of the subject based on an inspection data acquired based on a strength of the infrared light received by the first infrared light sensors and output a result of the inspection.

### CROSS-REFERENCE TO RELATED APPLICATIONS

This application is a Continuation Application of PCT Application No. PCT/JP2019/027824, filed Jul. 12, 2019, and based upon and claiming the benefit of priority from Japanese Patent Application No. 2018-142559, filed Jul. 30, 2018, the entire contents of all of which are incorporated herein by reference.

### FIELD

Embodiments described herein relate generally to a health condition inspection system.

### BACKGROUND

Generally, health condition check of people is performed by extracting and inspecting a fecal matter (for example, feces) discharged from a subject. In such inspection, finding occult blood in the feces may result in finding of colorectal cancer, for example.

However, in order to perform the above inspection, subjects themselves need to extract the fecal matter, and it is not easy. Thus, an easier system of inspect a health condition of a subject is desired.

### BRIEF DESCRIPTION OF THE DRAWINGS

FIG. 1 is a diagram of an example of a network of a health condition inspection system of a first embodiment.

FIG. 2 is a diagram of an example of system structure of a detection device.

FIG. 3 is a diagram illustrating an example of arrangement of an infrared light sensor unit formed in a sheet shape.

FIG. 4 is a diagram illustrating an example of system structure of an authentication device.

FIG. 5 is a diagram of an example of system structure of a server device.

FIG. 6 is a block diagram illustrating an example of function structure of the health condition inspection system.

FIG. 7 is a sequence chart illustrating an example of process sequence of the health condition inspection system.

FIG. 8 is a diagram illustrating an example of system structure of the authentication device if a subject is authenticated based on a finger print.

FIG. 9 is a block diagram illustrating an example of function structure of a health condition inspection system of a second embodiment.

FIG. 10 is a flowchart of an example of process sequence of a server device if a new trained model is prepared using an inspection result stored in storage.

### DETAILED DESCRIPTION

In general, according to one embodiment, a health condition inspection system includes a first light source, first infrared light sensors, and a processor. The first light source irradiates infrared light to a fecal matter of a subject, discharged into a toilet. The first infrared light sensors are arranged inside the toilet and configured to receive the infrared light irradiated from the first light source. The processor is configured to inspect a health condition of the subject based on an inspection data acquired based on a strength of the infrared light received by the first infrared light sensors and output a result of the inspection.

Various embodiments will be described hereinafter with reference to the accompanying drawings.

### First Embodiment

Initially, a first embodiment will be explained. FIG. 1 illustrates an example of network structure of a health condition inspection system of the present embodiment. The health condition inspection system of the present embodiment is used to inspect a health condition of a subject using a fecal matter discharged by the subject (target), for example.

As in FIG. 1, the health condition inspection system includes a detection device 10, authentication device 20, and server device (health condition inspection device) 30.

The detection device 10 is incorporated in, for example, a toilet disposed in a bathroom space, and is used to detect inspection data for inspecting the health condition of a subject based on a fecal matter of the subject discharged into the toilet.

The authentication device 20 is connected to the detection device 10, and is used to authenticate the subject. In the authentication device 20, a process to authenticate the subject is executed, and identification information to identify the subject (hereinafter, referred to as subject ID) is acquired.

Note that, in FIG. 1, one detection device 10 and one authentication device 20 are shown; however, the health condition inspection system may include a plurality of detection devices 10 and authentication devices 20 corresponding to the number of subjects using the health condition inspection system.

The inspection data acquired by the detection device 10 and the subject ID acquired by the authentication device 20 are transmitted from the authentication device 10 to the server device 30 via a network 40 such as the Internet, for example.

The server device 30 receives inspection data and subject ID transmitted from the authentication device 20. The server device 30 inspects a health condition of a subject identified by the subject ID based on the received inspection data.

Note that, in FIG. 1, the inspection data acquired by the detection device 10 is transmitted from the authentication device 20 to the server device 30; however, the inspection data may be transmitted from the detection device 10 which is connected to the network 40 to the server device 30. Similarly, the subject ID acquired by the authentication device 20 may be transmitted from the detection device 10 to the server device 30.

Furthermore, in the present embodiment, each of the detection device 10 and the authentication device 20 is an individual device for convenience of explanation; however, the detection device 10 and the authentication device 20 may be formed integrally with each other.

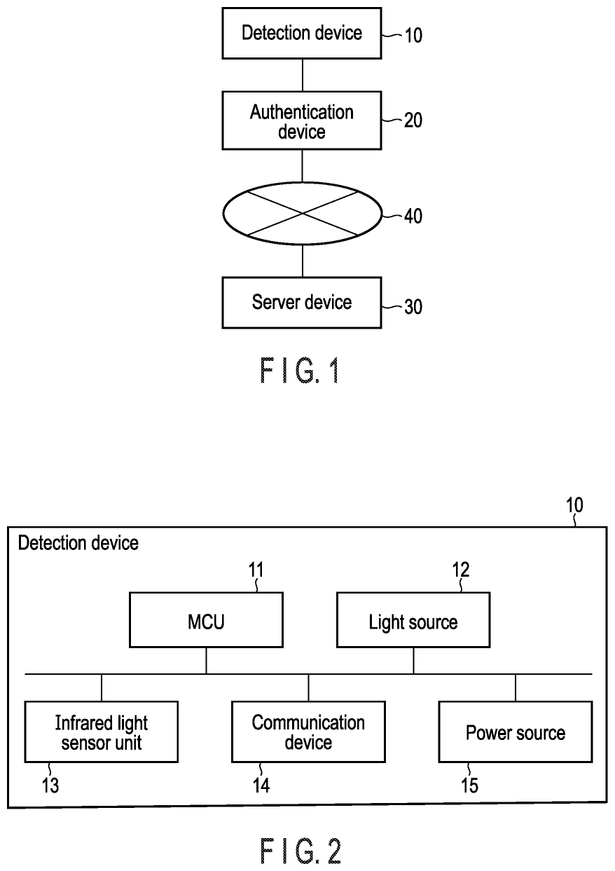

FIG. 2 illustrates an example of the system structure of the detection device 10 of FIG. 1. As in FIG. 2, the detection device 10 includes a micro control unit (MCU) 11, light source (first light source) 12, infrared light sensor unit 13, communication device 14, and power source 15, for example.

The MCU 11 is an incorporated microprocessor configured to control each of the components in the detection device 10. The MCU 11 executes software (program) such as firmware (F/W) stored in the detection device 10. Furthermore, the MCU 11 stores identification information (hereinafter referred to as detection device ID) to identify the detection device 10 (or a toilet to which the detection device 10 is incorporated).

The light source 12 is located to a position to irradiate infrared light to a fecal matter of a subject discharged into a toilet. The light source 12 may be arranged to the inner surface of the toilet or to a rear surface of a seat of the toilet (the opposite surface to the seating surface).

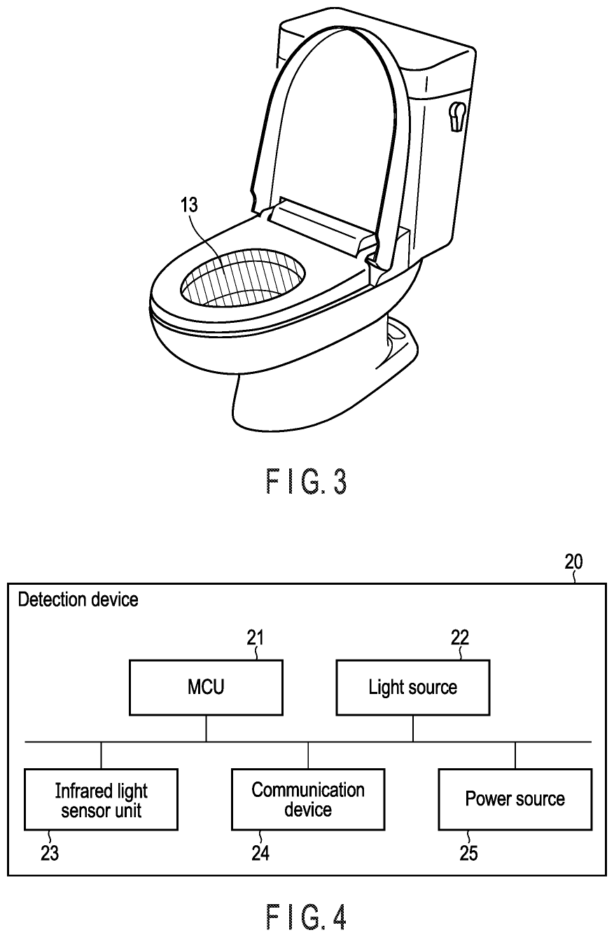

The infrared light sensor unit 13 includes a plurality of infrared light sensors (first infrared light sensors) to receive the infrared light irradiated from the light source 12. The infrared light sensor unit 13 (a plurality of infrared sensors) is formed in a sheet-like shape, and is arranged along (adhered to) the inner surface of the toilet as in FIG. 3. Note that, with the infrared light sensor unit 13 (infrared sheet sensor), the infrared light irradiated from the light source 12 which passing through the fecal matter in the toilet (transmitted light), or the infrared light reflected by the fecal matter (reflection light) can be received.

The communication device 14 is a device configured to execute communication with an external device through a wired or wireless manner, for example.

The power source 15 supplies power to the detection device 10. Each component in the detection device 10 can be operated with the power supplied from the power source 15.

FIG. 4 is an example of the system structure of the authentication device 20. In the authentication device 20 of the present embodiment, vein authentication is performed in which a subject is authenticated based on a vein pattern of the buttocks of the subject, for example.

As in FIG. 4, the authentication device 20 includes an MCU 21, light source 22, infrared light sensor unit 23, communication device 24, and power source 25, for example.

The MCU 21 is an incorporated microprocessor configured to control each component in the authentication device 20. The MCU 21 executes software (program) such as firmware (F/W) stored in the authentication device 20.

The light source 22 is located to a position to irradiate infrared light to the buttocks of a subject sitting on the seat of the toilet. The light source 22 is arranged on the seat surface of the seat.

The infrared light sensor unit 23 includes a plurality of infrared light sensors (second infrared light sensors) to receive infrared light irradiated from the light source 22. The infrared light sensor unit 23 is formed in a sheet-like shape, and is arranged on the seat surface of the sear with the light source 22, for example. The infrared light sensor unit 23 (infrared light sheet sensor) receives the infrared light irradiated by the light source 22 which is reflected by the buttocks of the subject (reflection light).

The communication device 24 is a device configured to execute communication with an external device in a wired or wireless manner, for example.

The power source 25 supplies power to the authentication device 20. Each component in the authentication device 20 is operated with the power supplied from the power source 25.

Note that, in FIGS. 2 and 4, each of the detection device 10 and the authentication device 20 includes a communication device and a power source; however, since the detection device 10 and the authentication device 20 are expected to be disposed in the same bathroom in the present embodiment, the MCU, communication device, and power source may be commonly used in both the detection device 10 and the authentication device 20.

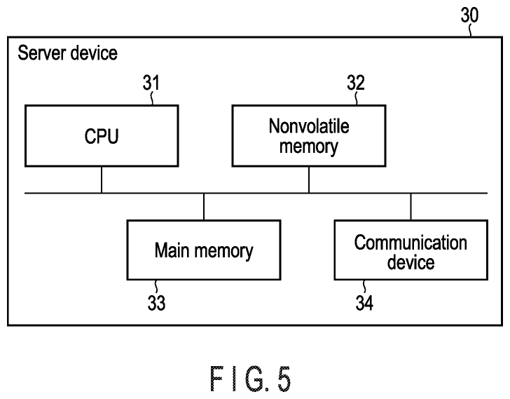

FIG. 5 illustrates an example of the system structure of the server device 30. As in FIG. 5, the server device 30 includes a CPU 31, nonvolatile memory 32, main memory 33, and communication device 34, for example.

The CPU 31 is a hardware processor configured to control each component in the server device 30. The CPU 31 executes various programs loaded from the nonvolatile memory 32 which is a storage device to the main memory 33. The programs executed by the CPU 31 include, for example, operating system (OS) and program to inspect the health condition of subjects. Furthermore, the CPU 31 executes, for example, basic input/output system (BIOS) which is a program for hardware control.

The communication device 34 is a device configured to execute communication with an external device in a wired or wireless manner.

Note that, in FIG. 5, only the CPU 31, nonvolatile memory 32, main memory 33, and communication device 34 are shown; however, other storage devices such as hard disk drive (HDD) and solid state drive (SDD) may be included, and an input device and an output device may further be included, for example.

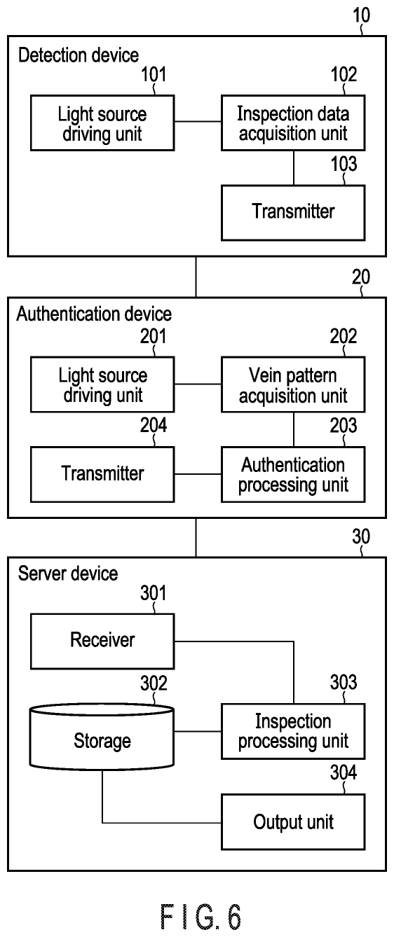

FIG. 6 is a block diagram illustrating an example of the function structure of the health condition inspection system of the present embodiment.

As in FIG. 6, the detection device 10 includes a light source driving unit 101, inspection data acquisition unit 102, and transmitter (transmitting unit) 103. In the present embodiment, a part or all of the units 101 to 103 included in the detection device 10 may be achieved by the MCU 11 executing programs, that is, by means of software, or may be achieved by means of hardware, or may be achieved by a combination of such software and hardware.

The light source driving unit 101 drives the light source 12 of the detection device 10 when inspecting the health condition of subjects in the health condition inspection system. When the light source driving unit 101 drives the light source 12, the infrared light is irradiated from the light source 12 to the fecal matter in the toilet.

The inspection data acquisition unit 102 acquires, when the infrared light is irradiated to the fecal matter as described above, inspection data based on a strength of the infrared light received by the infrared light sensor unit 13 of the detection device 10. The inspection data acquired by the inspection data acquisition unit 102 represents distribution of the strength of the infrared light received by each of the infrared light sensors of the infrared light sensor unit 13, for example.

Note that absorption rates of infrared light by the fecal matter and blood components (hemoglobin in blood) are different from that of other objects (substances) in the toilet, and there are differences in infrared components included in the light (reflected light or transmitted light) from the fecal matter and blood components. Thus, according to the distribution of the strength of infrared light received by each of the infrared light sensors when the infrared light is irradiated to an area including the fecal matter, the inspection data corresponding to an image representing a shape of the fecal matter and blood components mixed in the fecal matter can be acquired.

Note that the infrared light sensors included in the infrared light sensor unit 13 can acquire signals representing the strength of the infrared light received by the infrared light sensors, and the inspection data is acquired by processing the signals through, for example, an analogue front end circuit (A/D converter).

The transmitter 103 transmits the inspection data acquired by the inspection data acquisition unit 102 to the authentication device 20 via the communication device 14.

As in FIG. 6, the authentication device 20 includes a light source driving unit 201, vein pattern acquisition unit 202, authentication processing unit 203, and transmitter transmission unit) 204. In the present embodiment, a part or all of the units 201 to 204 included in the authentication device 20 may be achieved by the MCU 21 executing programs, that is, by means of software, or may be achieved by means of hardware, or may be achieved by a combination of such software and hardware.

The light source driving unit 201 drives the light source 22 of the authentication device 20 when a subject sits on the toilet seat. When the light source 22 is driven by the light source driving unit 201, infrared light is irradiated from the light source 22 to the buttocks of the subject sitting on the seat.

When the infrared light is irradiated to the buttocks of the subject as above, the vein pattern acquisition unit 202 acquires (reads) the vein pattern of the buttocks of the subject based on the strength of the infrared light received by the infrared light sensor unit 23 of the authentication device 20. The vein pattern acquired by the vein pattern acquisition unit 202 represents distribution of the strength of the infrared light received by each of the infrared light sensors of included in the infrared light sensor unit 23, for example.

Here, since the absorption rate of the infrared light of the blood components is higher than that of other living body components, according to the distribution of the strength of the infrared light received by each of the infrared light sensors when the infrared light is irradiated to the buttocks of the subject, the vein pattern representing the vein through which the blood components flow in the buttocks can be acquired.

Note that the infrared light sensors included in the infrared light sensor unit 23 can acquire signals representing the strength of the infrared light received by the infrared light sensors, and the vein pattern is acquired by processing the signals through, for example, an analogue front end circuit (A/D converter).

The authentication processing unit 203 executes an authentication process based on the vein pattern acquired by the vein pattern acquisition unit 202. Through the authentication process, a subject ID to identify the subject corresponding to the vein pattern acquired by the vein pattern acquisition unit 202 can be acquired.

The transmitter 204 transmits the subject ID acquired by the authentication processing unit 203 and the inspection data transmitted from the detection device 10 (transmitter 103) to the server device 30 via the communication device 24.

As in FIG. 6, the server device 30 includes a receiver (receiving unit) 301, storage 302, inspection processing unit 303, and output unit 304. In the present embodiment, a part or all of the units 301, 303, and 304 included in the server device 30 may be achieved by the MCU 21 executing programs, that is, by means of software, or may be achieved by means of hardware, or may be achieved by a combination of such software and hardware. Furthermore, the storage 302 may be realized as a nonvolatile memory 32 of the server device 30, or may be realized as a different storage device.

The receiver 301 receives the subject ID and the inspection data from the authentication device 20 (transmitter 204) via the communication device 34.

Here, the storage 302 preliminarily stores an inspection model used when the health condition of subjects is inspected. The inspection model corresponds to a trained model prepared by learning learning data including inspection data based on strength of infrared light irradiated to fecal matters of other subjects (for example, subjects including healthy subjects and unhealthy subjects) (that is, inspection data of other subjects) and the health condition of the other subjects. In other words, the inspection model includes an algorithm defining a relationship between the inspection data and the health condition, and by inputting the inspection data thereto, the health condition corresponding to the inspection data can be output.

Note that the inspection model may be prepared in the server device 30 by acquiring many learning data, for example, or may be prepared in an external device.

The inspection processing unit 303 inspects the health condition of the subject using the inspection model stored in the storage 302. Specifically, the inspection processing unit 303 inputs the inspection data received by the receiver 301 to the inspection model and acquires a health condition corresponding to the inspection data output from the inspection model (that is, health condition of the subject) as an inspection result.

Note that the health condition of the subject inspected by the inspection processing unit 303 includes, for example, presence/absence (finding/no finding) of blood component in the fecal matter of the subject (that is, occult blood in feces).

The health condition of the subject acquired by the inspection processing unit 303 (that is, the inspection result from the inspection processing unit 303) is output to an external device, for example, by the output unit 304. Note that the inspection result from the inspection processing unit 303 may be stored in the storage 302 with the aforementioned subject ID and inspection data.

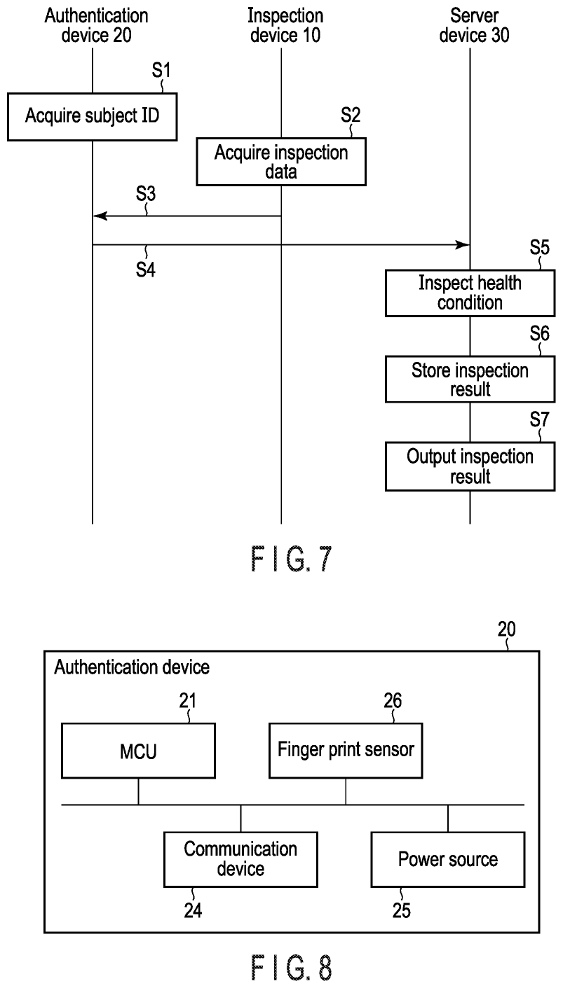

Hereinafter, an example of a process sequence of the health condition inspection system of the present embodiment will be explained with reference to the sequence chart of FIG. 7. Note that, the process depicted in FIG. 7 may be executed at each time when a subject uses a toilet, or may be executed at a predetermined period (for example, once per day).

Initially, a case where a subject sits on the seat of the toilet situated in a bathroom to discharge a fecal matter such as feces is assumed.

In that case, an authentication process to authenticate the subject is executed in the authentication device 20. In the authentication process, the light source 22 configured to irradiate infrared light to the buttocks of the subject sitting on the seat is driven by the light source driving unit 201, and thus, the vein pattern (vein data) of the subject based on a strength of the infrared light received by the infrared light sensor unit 23 is acquired.

Here, in the authentication processing unit 203 (or in the authentication device 20), for example, the vein pattern per subject using the health condition inspection system is preliminarily registered, and the subject ID to identify the subject corresponding to the vein pattern is held.

Through the above, the authentication processing unit 203 can acquire the subject ID held in associated with the vein pattern matching the acquired vein pattern (step S1).

In this example, the subject ID is held in the authentication processing unit 203 as explained above; however, the subject ID may be managed in and acquired from the server device 30 or a difference external device, for example.

Then, when the subject sitting on the seat discharges the fecal matter, the detection device 10 acquires the inspection data (step S2).

In step S2, the light source 12 to irradiate the infrared light to the fecal matter discharged into the toilet is driven by the light source driving unit 101, and thus, the inspection data based on a strength of the infrared light received by the infrared light sensor unit 13 is acquired. Note that the inspection data acquired in step S2 is, as described above, the data corresponding to an image representing the shape of fecal matter of the subject, blood components in the fecal matter and the like.

When the process of step S2 is executed, the inspection data acquired in step S2 (inspection data of subject) is transmitted from the detection device 10 (transmitter 103) to the authentication device 20 (step S3).

The authentication device 20 (transmitter 204) transmits the subject ID acquired in step S1 and the inspection data transmitted in step S3 (inspection data acquired in step S2) to the server device 30 (step S4).

Note that, in this example, the authentication device 20 is communicably connected to the server device 30 via the network 40 as in FIG. 1; however, if the detection device 10 is communicably connected to the server device 30 via the network 40, the subject ID and the inspection data may be transmitted to the server device 30 from the detection device 10.

When the process of step S4 is executed, the subject ID and the inspection data transmitted in step S4 are received by the receiver 301 of the server device 30.

Then, the inspection processing unit 303 inspects the health condition of the subject based on the inspection data received by the receiver 301 (step S5). In step S5, the inspection processing unit 303 acquires the health condition output from the inspection model as the inspection data being input to the inspection model stored in the storage 302 as the health condition of the subject. Note that, in the present embodiment, the health condition of the subject acquired by the inspection processing unit 303 includes, for example, presence/absence of occult blood in the fecal matter (for example, feces).

When the process of step S5 is executed, the inspection result from the inspection processing unit 303 (the health condition of the subject acquired by the inspection processing unit 303) is associated with the subject ID and the inspection data received by the receiver 301 and is stored in the storage 302 (step S6). Note that, in this example, the subject ID, inspection data, and inspection result are stored in the storage 302; however, date when the subject ID and the inspection data are received (that is, inspection date) and the like may be stored in the storage 302 in addition to the subject ID, inspection data, and inspection result.

Furthermore, as described above, the detection device ID held in the MCU 11 of the detection device 10 may be stored in the storage 302. In that case, the detection device ID is transmitted with inspection data from the detection device 10 to the authentication device 20 in step S3, and the subject ID, the inspection data and the detection device ID are transmitted from the authentication device 20 to the server device 30 in step S4.

Then, the output unit 304 outputs, for example, the subject ID and inspection result stored in the storage 302 (step S7).

Note that, in step S7, the subject ID and the inspection result may be output to a display or the like to be viewed by a manager of the health condition inspection system, for example.

Furthermore, the subject ID and the inspection result may be output (transmitted) to the authentication device 20, for example. In that case, the subject ID and the inspection result may be output to, for example, a display connected to the authentication device 20 (display in the bathroom) for viewing.

In this example, the subject ID and the inspection result are to be output; however, as mentioned above, if the date of inspection and the inspection device ID are stored in the storage 302, the date of inspection and the inspection device ID may be output with the subject ID and the inspection result.

Furthermore, processes of steps S1 to S7 in FIG. 7 are executed as a sequence in this example; however, the subject ID and the inspection data transmitted from the authentication device 20 may be accumulated in the storage 302 in step S4 and the process of inspecting the health condition of the subject may be executed later. That is, the processes of steps S1 to S4 and the processes of steps S5 to S7 in FIG. 7 may be executed separately.

Although this is not mentioned in the description of FIG. 7, there may be a motion sensor in a bathroom. With such a motion sensor, a person (subject) entering the bathroom can be detected. Furthermore, there may be a pressure sensor on a seat of a toilet. With such a pressure sensor, a person (subject) sitting on the seat can be detected based on a weight or the like on the seat.

The health condition inspection system of the present embodiment may be structured to cooperate (associated) with the motion sensor and the pressure sensor.

In that case, the health condition inspection system is activated when a subject entering a bathroom is detected by the motion sensor, and the detection device 10 is turned on at that time.

Furthermore, when the subject sitting on the seat is detected by the pressure sensor, the authentication device 20 may be turned on, for example.

As described above, with the health condition inspection system cooperating with the motion sensor and the pressure sensor, the power used by the health condition inspection system can be reduced.

As described above, in the present embodiment, the infrared light is irradiated to a fecal matter of a subject discharged into a toilet to acquire inspection data based on a strength of the infrared light received by the infrared light sensor unit 13 (first infrared light sensors) arranged inside the toilet, and a health condition of the subject is inspected based on the inspection data. In the present embodiment, with such a structure, health conditions of the subject can easily be inspected.

Furthermore, in the present embodiment, the infrared light sensor unit 13 is formed in a sheet-like shape to be along with (adhered to) the inner surface of the toilet. With such a structure, proper inspection data can be acquired (detected) while the inspection to the fecal matter is oblivious to the subject.

Note that, when the inspection data is acquired in the detection device 10, the subject sits on the seat, and thus, there may be insufficient light inside the toilet. Thus, when acquiring the inspection data, a time to receive the infrared light (that is, time of exposure) should be set long in the infrared light sensor unit 13.

Furthermore, in the present embodiment, the infrared light sensor unit 13 of the detection device 10 is formed in a sheet-like shape to be adhered to the inner surface of the toilet; however, the infrared light sensors included in the infrared light sensor unit 13 may be embedded in the inner surface of the toilet, or may be arranged at difference positions of the toilet as long as the infrared light irradiated to a fecal matter in the toilet can sufficiently be received.

Furthermore, in the present embodiment, since a health condition of a subject is inspected using an inspection model prepared based on leaning data including inspection data of other subjects (inspection data based on strengths of infrared light irradiated to fecal matters of other subjects) and the health conditions of the other subjects, the inspection result with higher accuracy can be achieved. Furthermore, in the present embodiment, since presence/absence of occult blood in feces can be detected, for example, finding of colorectal cancer can be facilitated.

Note that the health condition of the subject inspected by the health condition inspection system of the present embodiment is not limited to finding occult blood in feces (fecal matter) of the subject. Specifically, whether or not feces of a subject is soft (that is, if the fecal matter is solid or is loose) may be inspected to determine the health condition of the subject. In that case, an inspection model to output whether or not feces of the subject is soft (if the feces is liquefied or solid) based on the inspection data of the subject is prepared in the storage 302 of the server device 30, for example, and whether or not the feces of the subject is soft can be inspected using the inspection model.

Furthermore, from inspection data achieved from a state where feces is in a toilet (that is, feces remains in the toilet), it may be difficult to accurately determine whether or not the feces is soft. In such a case, inspection data may be acquired in a period of time from discharging the feces from the subject to the feces dropping onto the water (that is, during a dropping time) at a sampling period corresponding to a motion picture of 15 Hz or more, for example. With such an inspection data, whether or not the feces is soft can be determined with relatively high accuracy.

In this example, whether or not the feces is soft is determined; however, the amount of feces of the subject or the like may be inspected based on the inspection data, for example.

Furthermore, in the present embodiment, the authentication device 20 configured to authenticate a subject and to acquire subject ID (identification information) to identify the subject is used, and the subject ID acquired by the authentication device 20 and the inspection result are output. Note that, in the present embodiment, the subject ID and the inspection result are output to a manager terminal used by a manager of the health condition inspection system to be presented to the manager, for example. Thus, the manager can check the health condition (inspection result) of the subject identified by the subject ID. Furthermore, in the present embodiment, the subject ID and the inspection result may be output to a display or the like provided with the bathroom to be presented (notified) to the subject, for example. Thus, the subject can check the health condition (inspection result) of oneself. Note that the subject ID and the inspection result may be notified to the subject through a voice or illumination, for example.

Furthermore, the subject ID and the inspection result may be output to an external system operated by an insurance company, for example. Through this step, the inspection result may be considered in, for example, calculation of insurance fee of the subject identified by the subject ID. Note that the subject ID and the inspection result of the present embodiment may be used in other services (systems). Furthermore, the subject ID and the inspection result may be accumulated in the storage 302 with the inspection data to be used in various analytic purposes.

Furthermore, in relation to the authentication process of the authentication device 20, the authentication device 20 includes a light source 22 (second light source) to irradiate infrared light to buttocks of a subject sitting on a seat of a toilet, and an infrared light sensor unit 23 (a plurality of second infrared light sensors) arranged to the seat surface of the seat to receive the infrared light irradiated from the light source 22, and acquires a subject ID to identify the subject based on a vein pattern of the subject based on a strength of the infrared light received by the infrared light sensor unit 23.

In the present embodiment, with the aforementioned structure, the subject can be identified (authenticated) based on an ordinary movement of the subject discharging a fecal matter (that is, using the bathroom).

Note that, in the present embodiment, a subject is authenticated based on the vein pattern of the buttocks of the subject by the authentication device 20; however, the authentication device 20 may be structured to authenticate the subject based on a finger print. In that case, the authentication device 20 may include a finger print sensor 26 to acquire finger print data representing the finger print of the subject as in FIG. 8.

Here, in order to flush out the fecal matter discharged in the toilet, a subject operates an operation unit including a predetermined button (for example, flush button). In that case, the finger print sensor 26 of the authentication device 20 is provided with a position to cover the button to flush out the fecal matter (that is, a positon where a finger of the subject touches to operate the operation unit).

Thus, the finger print sensor 26 can acquire finger print data representing the finger print of the subject when the subject operates the operation unit, and the authentication processing unit 203 of the authentication device 20 can acquire the subject ID to identify the subject corresponding to the acquired finger print.

With this structure, the subject can be identified (authenticated) based on an ordinary movement of the subject discharging a fecal matter (that is, using the bathroom) even if the subject is identified based on the finger print by the authentication device 20.

Note that, if the subject is authenticated by the authentication device 20 and the subject ID to identify the subject is acquired as above, a setting of toilet such as Washlet (registered trademark) may be automatically changed depending on the subject identified by the subject ID, for example.

Furthermore, in the present embodiment, the detection device 10 is incorporated with the toilet; however, the detection device 10 may be attached to a pipe to flush out the fecal matter of the subject discharged into the toilet to the outside (drain pipe). In that case, the light source 12 and the infrared light sensor unit 13 of the detection device 10 may be arranged inside the pipe, for example. With this structure, infrared light is irradiated to the fecal matter passing through the pipe from the light source 12, and the infrared light (transmitted light or reflection light) is received by the infrared light sensor unit 13 to acquire the inspection data.

Note that, if the detection device 10 is attached to the pipe, as compared to the structure where the detection device 10 is incorporated with the toilet (the infrared light sensor unit 13 is arranged inside the toilet), the influence of external light noise with respect to the inspection data can be decreased. Furthermore, if the detection device 10 is attached to the pipe, inspection data (image) including the shape of the fecal matter passing through the pipe or the like can be acquired, and thus, whether or not feces is soft can be inspected with relatively higher accuracy.

Note that the health condition inspection system of the present embodiment may be structured to include a plurality of detection devices 10. For example, a first detection device 10 may be incorporated with the toilet, and a second detection device 10 may be attached to the pipe. With multiple detection devices 10, inspection data with higher accuracy can be achieved.

Furthermore, in the present embodiment, if the detection device 10 is incorporated with the toilet or attached to a pipe, power supply to the detection device 10 may become difficult. In that case, for example, the detection device 10 may include a wireless power supply antenna to enable wireless power charge to the detection device 10.

Furthermore, the inspection data is acquired based on the fecal matter of the subject in order to inspect the health condition of the subject, and in addition thereto, various sensors to measure pulse and blood glucose level of the subject may be provided with the seat of the toilet on which the subject sits and a handrail which is held by the subject, for example. With such a structure, data of pulse and blood glucose level can be acquired with the inspection data based on the fecal matter.

In this example, when the detection device 10 is shipped, manufacturing process data and RAW data of the detection device 10 are stored in the server device 30, for example. Note that the manufacturing process data includes a factory ID to identify a factory where the detection device 10 is manufactured and information related to a route of the detection device 10 manufactured, for example. Furthermore, if the detection device 10 is incorporated with the toilet, RAW data includes factory-setting inspection data acquired by the detection device 10 while there is no fecal matter in the toilet. On the other hand, if the detection device 10 is attached to the pipe, RAW data includes factory-setting inspection data acquired by the detection device 10 while there is no fecal matter in the pipe.

The server device 30 stores the manufacturing process data and the RAW data as being associated with the detection device ID held inside the MCU 11 of the detection device 10. In that case, the detection device 10 is identified by the detection device ID held in the MCU 11, and the manufacturing process data and the RAW data of the detection device 10 can be specified (acquired).

The manufacturing process data of the detection device 10 can be used, if there is a malfunction in the detection device 10, for analyzing a cause of the malfunction, for example.

Furthermore, RAW data of the detection device 10 can be used for correction of detection data (image) acquired (detected) in the detection device 10. Specifically, using RAW data, influence of unevenness in the brightness occurring in RAW data can be suppressed in the inspection data.

Furthermore, if the correction of inspection data using RAW data exceeds a predetermined value, a feedback may be sent to the manufacturer, or designer, or the like of the detection device 10 to notify a possibility of malfunction in the detection device 10. Furthermore, by comparing RAW data acquired by the detection device 10 at the shipping time and RAW data acquired by the detection device 10 after predetermined days or years, malfunction or the like caused by aging of the detection device 10 may be detected, and a feedback of result of such detection may be sent to the manufacturer, or designer, or the like of the detection device 10.

Furthermore, in order to improve the quality of inspection data (image) acquired by the detection device 10, several inspection data may be acquired from a fecal matter of a subject, and a health condition of the subject may be inspected using the inspection data least affected by the noise from the several inspection data. Furthermore, a noise canceling process of averaging the several inspection data may be executed, for example.

Furthermore, in the present embodiment, the fecal matter of the subject is feces; however, the fecal matter of the subject may be urine. Furthermore, if a subject sitting on a seat is not detected by the pressure sensor and an object is detected in a toilet based on inspection data, a type of the object (which may be, for example, urine discharged by a male subject, or vomit, or something accidentally dropped therein) may be determined using a preliminarily prepared trained model to execute different processes corresponding to results of determination.

Furthermore, in the present embodiment, the light source 12 of the detection device 10 irradiates infrared light; however, light other than infrared light (infrared region), which can be used to inspect (determine) presence/absence of occult blood (blood components) in feces as described above may be used. Note that, in a similar manner, the light source 22 of the authentication device 20 to authenticate a subject based on the vein pattern of the buttocks of the subject may use light other than infrared light which can be used to acquire (read) the vein pattern of the buttocks.

Furthermore, in the present embodiment, the light source 12 of the detection device 10 is arranged in the inner surface of the toilet or in the rear surface of the toilet seat; however, the light source 12 may be incorporated with the infrared light sensor unit 13, or may be embedded in the inner surface of the toilet seat.

Note that, in the present embodiment, the health condition of the subject is inspected in the server device 30; however, a process of inspecting the health condition of the subject may be executed in the detection device 10 side, or may be executed in the authentication device 20 side, for example.

Furthermore, in the present embodiment, the health condition inspection system includes the detection device 10, authentication device 20, and server device 30; however, the health condition inspection system may be realized as a single device including all functions of the detection device 10, authentication device 20, and server device 30, for example.

### Second Embodiment

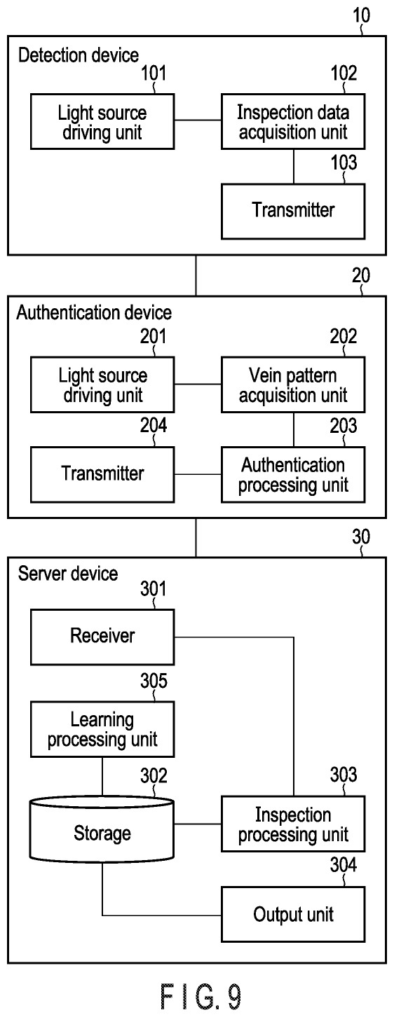

Now, the second embodiment will be explained. FIG. 9 is a block diagram illustrating an example of the functional structure of a health condition inspection system of the present embodiment. Note that elements similar to those of FIG. 6 are referred to by the same reference numbers and their detailed descriptions will be omitted. In this example, elements different from those of FIG. 6 will be mainly explained.

Furthermore, the network structure, and the system structures of the detection device, authentication device, and server device of the health condition inspection system of the present embodiment are the same as those of the first embodiment. Thus, they will be explained with reference to FIGS. 1 to 5.

The present embodiment includes a server device 30 with a learning processing unit 305 as in FIG. 9, and in this respect, it is different from the first embodiment. Note that, in the present embodiment, the learning processing unit 305 may be realized by the CPU 31 executing programs, that is, by means of software, or may be realized by means of hardware, or may be realized as a combination of such software and hardware.

Note that, storage 302 of the server device 30 preliminarily stores an inspection model used to inspect a health condition of a subject, as described in the aforementioned first embodiment. Furthermore, if the processes of FIG. 7 are executed, the storage 302 stores, for example, a subject ID, inspection data, and inspection result.

In the present embodiment, the learning processing unit 305 uses the inspection data and the inspection result stored in the storage 302 to update the inspection model stored in the storage 302.

Note that, as described above, the inspection model is a trained model prepared by learning the learning data including (a pair of) inspection data of other subjects and health conditions, and the learning processing unit 305 updates the inspection model by learning a pair of the inspection data and the inspection result associated with subject IDs and stored in the storage 302 (that is, the inspection data acquired in step S2 and result inspected in step S5 of FIG. 7) as new learning data.

In the present embodiment, the inspection model is updated as above, and thus, the accuracy of the inspection model (accuracy of inspection of health condition of subjects using the inspection model) can be improved.

In this example, the inspection data and the inspection result stored in the storage 302 are learnt as the learning data, and if the accuracy of the inspection model is low, the inspection result may be an error. In that case, for example, the health condition inspection system may be cooperated with a system of a medical organization such as a hospital used by the subject identified by the subject ID stored in the storage 302 such that a diagnosis of the subject by the medical organization can be reflected to the inspection result of the subject stored in the storage 302.

In that case, the learning processing unit 305 can learn using learning data including the inspection data and diagnosis to the subject (that is, proper inspection result) (that is, the learning processing unit 305 can perform reinforced learning), and thus, the accuracy of the inspection model can further be improved.

Note that, the inspection result stored in the storage 302 may be used for the improvement of accuracy of already-available inspection model (for example, inspection model used to inspect presence/absence of occult blood in feces) as described above; however, it may be used for preparing a new trained model.

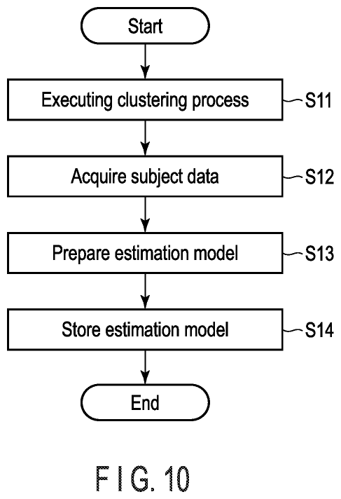

Hereinafter, a series of processes performed by the server device 30 to prepare a new trained model using the inspection result stored in the storage 302 in the present embodiment will be explained with reference to the flowchart of FIG. 10. Note that the process of FIG. 10 is executed by the learning processing unit 305.

The aforementioned process of FIG. 7 is executed per subject using the health condition inspection system. Thus, the storage 302 stores (accumulates) subject IDs to identify the subjects, inspection data of each subject, and inspection result of each subject therein. Note that the storage 302 may store, for example, subject IDs collected (registered) beforehand in medical organizations and the like, inspection data and inspection results of the subjects.

Initially, the learning processing unit 305 executes a clustering process with respect to the multiple inspection data stored in the storage 302 (step S11). Through the clustering process executed in step S11, inspection data having the same or similar features can be classified as the same category, for example. In that case, by executing an image processing with respect to the inspection data (images), the inspection data indicating similar amount and distribution of occult blood can be classified to the same category.

Then, the learning processing unit 305 acquires data of health condition of each subject identified by the subject ID stored in the storage 302 while being associated with each of the inspection data classified as the same category in step S11 (hereinafter referred to as subject data) (step S12). The subject data includes a medical history of the subject, and may further include a blood type, age, and sex of the subject. The subject data is acquired from a system of a medical organization cooperating with the health condition inspection system (that is, from the outside of the health condition inspection system) as described above. Note that the subject data may be managed in the server device 30 or the like.

When the process of step S12 is executed, the learning processing unit 305 sequentially learns a pair of the inspection data classified as the same category in step S11 and the subject data acquired in step S12 (learning data), and prepares a trained model used for estimation of the health condition of the subject (hereinafter referred to as estimation model) (step S13).

The estimation model prepared in step S13 is stored in the storage 302, for example (step S14).

Through the aforementioned process of FIG. 10, an estimation model including an algorithm defining relationship between the inspection data of subject and health condition based on the subject data of the subject, which can output the health condition corresponding to the inspection data in response to input of the inspection data.

That is, according to the estimation model, for example, a subject from which the inspection data similar to the inspection data classified to the same category in step S11 is acquired may be considered to have a tendency to have a disease (illness) which is included in the subject data acquired in step S12 as medical history, and thus, a possible disease the subject may have can be estimated, for example.

As described above, in the present embodiment, the inspection data stored in the storage 302 and the inspection result are learnt as a learning data, and the inspection model stored in the storage 302 is updated, and thus, the accuracy of the inspection model can be improved at each time when the inspection of the health condition of the subject is performed.

Furthermore, in the present embodiment, the inspection data stored in the storage 302 is classified, subject data related to the health condition of the subject identified by the subject ID stored in the storage 302 while being associated with the classified inspection data is acquired, and an estimation model to estimate the health condition of the subject based on the learning data including the classified inspection data and the acquired subject data is prepared. With such a structure, a possible disease the subject may have can be estimated from the inspection data using the prepared estimation model, and thus, a warning to the subject who uses the health condition inspection system for inspection of the health condition can be made, for example.

That is, the inspection data acquired in the health condition inspection system of the present embodiment can further be effectively used by being compared to other big data (such as subject data), for example.

Hereinafter, the inventions of the embodiments will be cited.

[C1]

A health condition inspection system, including:
a first light source to irradiate infrared light to a fecal matter of a subject, discharged into a toilet;
a plurality of first infrared light sensors arranged inside the toilet and configured to receive the infrared light irradiated from the first light source; and
a processor configured to:
inspect a health condition of the subject based on an inspection data acquired based on a strength of the infrared light received by the first infrared light sensors; and
output a result of the inspection.

[C2]

The health condition inspection system of [C1], wherein the first infrared light sensors are shaped as a sheet to be along with the inner surface of the toilet.

[C3]

The health condition inspection system of [C1], wherein the processor is configured to inspect the health condition of the subject using an inspection model prepared based on inspection data acquired based on a strength of infrared light irradiated to a fecal matter of a different subject and learning data including a health condition of the different subject.

[C4]

The health condition inspection system of [C3], wherein
the fecal matter includes feces, and
the result of the inspection includes finding/no finding of occult blood in the feces.

[C5]

The health condition inspection system of [C3], wherein the processor is configured to update the inspection model based on learning data including the inspection data acquired based on the strength of the infrared light received by the first infrared light sensors and the result of the inspection.

[C6]

The health condition inspection system of [C1], wherein the processor is configured to:
authenticate the subject and to acquire identification information to identify the subject, and
output the acquired identification information and the result of the inspection.

[C7]

The health condition inspection system of [C6], further including:
a second light source configured to irradiate infrared light to buttocks of the subject sitting on a toilet seat attached to the toilet; and
a plurality of second infrared light sensors arranged on the seat surface of the toilet seat to receive the infrared light irradiated from the second light source, wherein
the processor is configured to acquire the identification information to identify the subject based on a vein pattern of the subject acquired based on a strength of the infrared light received by the second infrared light sensors.

[C8]

The health condition inspection system of [C6], further including:
an operation unit operated by the subject to flush out the fecal matter discharged into the toilet; and
a finger print sensor provided with a position where a finger of the subject touches when the subject operates the operation unit to acquire finger print data indicative of the finger print of the subject, wherein
the processor is configured to acquire the identification information to identify the subject based on the finger print data acquired by the finger print sensor.

[C9]

The health condition inspection system of [C6], further including storage configured to associate a plurality of subjects including the subject with identification information to identify each subject and stores inspection data acquired based on a strength of infrared light irradiated to the fecal matter of the subject,
wherein the processor is configured to:
classify the inspection data stored in the storage,
acquire subject data related to a health condition of a subject identified by the identification information stored in the storage as being associated with the classified inspection data, and
prepare an estimation model to estimate the health condition of the subject based on the learning data including the classified inspection data and the acquired subject data.

[C10]

A health condition inspection system including:
a light source arranged inside a pipe to flush out a fecal matter of a subject discharged into a toilet, the light source configured to irradiate infrared light to the fecal matter passing through the pipe;
a plurality of infrared light sensors arranged inside the pipe to receive the infrared light irradiated by the light source;
a processor configured to:
inspect a health condition of the subject based on inspection data acquired based on a strength of the infrared light received by the infrared light sensors; and
output a result of the inspection.

[C11]

A health condition inspection system including:
an inspection device incorporated with a toilet; and
a server device connected to the detection device in a communicable manner, wherein
the detection device includes;
a light source configured to irradiate infrared light to a fecal matter of a subject discharged into the toilet; and
a plurality of infrared light sensors arranged inside the toilet to receive the infrared light irradiated by the light source, and
the server device includes;
a processor configured to inspect a health condition of the subject based on inspection data acquired based on a strength of the infrared light received by the infrared light sensors, and output a result of the inspection.

[C12]

A detection device incorporated with a toilet, the device including:
a light source to irradiate infrared light to a fecal matter of a subject discharged into the toilet;
a plurality of infrared light sensors arranged inside the toilet to receive the infrared light irradiated by the light source; and
a processor configured to acquire inspection data based on a strength of the infrared light received by the infrared light sensors, wherein
the inspection data is used to inspect a health condition of the subject.

[C13]

A health condition inspection device communicably connected to a detection device including a light source to irradiate infrared light to a fecal matter of a subject discharged into a toilet and a plurality of infrared light sensors to receive the infrared light irradiated from the light source, the health condition inspection device including:
a processor configured to:
inspect a health condition of the subject based on inspection data acquired based on a strength of the infrared light received by the infrared light sensors; and
output a result of the inspection.

[C14]

A method including:
irradiating infrared light to a fecal matter of a subject discharged into a toilet;
receiving the irradiated infrared light by a plurality of infrared light sensors arranged inside the toilet;
inspecting a health condition of the subject based on inspection data acquired based on a strength of the infrared light received by the infrared light sensors; and
outputting a result of the inspection.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A health condition inspection system comprising:
a first light source to irradiate infrared light to a fecal matter of a subject, discharged into a toilet;
a plurality of first infrared light sensors arranged inside the toilet and configured to receive the infrared light irradiated from the first light source; and
a processor configured to:
inspect a health condition of the subject based on an inspection data acquired based on a strength of the infrared light received by the first infrared light sensors; and
output a result of the inspection.

2. The health condition inspection system of claim 1, wherein the first infrared light sensors are shaped as a sheet to be along with the inner surface of the toilet.

3. The health condition inspection system of claim 1, wherein the processor is configured to inspect the health condition of the subject using an inspection model prepared based on inspection data acquired based on a strength of infrared light irradiated to a fecal matter of a different subject and learning data including a health condition of the different subject.

4. The health condition inspection system of claim 3, wherein
the fecal matter includes feces, and
the result of the inspection comprises finding/no finding of occult blood in the feces.

5. The health condition inspection system of claim 3, wherein the processor is configured to update the inspection model based on learning data comprising the inspection data acquired based on the strength of the infrared light received by the first infrared light sensors and the result of the inspection.

6. The health condition inspection system of claim 1, wherein the processor is configured to:
authenticate the subject and to acquire identification information to identify the subject; and
output the acquired identification information and the result of the inspection.

7. The health condition inspection system of claim 6, further comprising:
a second light source configured to irradiate infrared light to buttocks of the subject sitting on a toilet seat attached to the toilet; and
a plurality of second infrared light sensors arranged on the seat surface of the toilet seat to receive the infrared light irradiated from the second light source, wherein
the processor is configured to acquire the identification information to identify the subject based on a vein pattern of the subject acquired based on a strength of the infrared light received by the second infrared light sensors.

8. The health condition inspection system of claim 6, further comprising:
an operation unit operated by the subject to flush out the fecal matter discharged into the toilet; and
a finger print sensor provided with a position where a finger of the subject touches when the subject operates the operation unit to acquire finger print data indicative of the finger print of the subject, wherein
the processor is configured to acquire the identification information to identify the subject based on the finger print data acquired by the finger print sensor.

9. The health condition inspection system of claim 6, further comprising storage configured to associate a plurality of subjects including the subject with identification information to identify each subject and store inspection data acquired based on a strength of infrared light irradiated to the fecal matter of the subject, wherein
the processor is configured to:
classify the inspection data stored in the storage;
acquire subject data related to a health condition of a subject identified by the identification information stored in the storage as being associated with the classified inspection data; and
prepare an estimation model to estimate the health condition of the subject based on the learning data including the classified inspection data and the acquired subject data.

10. A health condition inspection system comprising:
a light source arranged inside a pipe to flush out a fecal matter of a subject discharged into a toilet, the light source configured to irradiate infrared light to the fecal matter passing through the pipe;
a plurality of infrared light sensors arranged inside the pipe to receive the infrared light irradiated by the light source; and
a processor configured to:
inspect a health condition of the subject based on inspection data acquired based on a strength of the infrared light received by the infrared light sensors; and
output a result of the inspection.

11. A health condition inspection system comprising:
an inspection device incorporated with a toilet; and
a server device connected to the detection device in a communicable manner, wherein
the detection device comprises:
a light source configured to irradiate infrared light to a fecal matter of a subject discharged into the toilet; and
a plurality of infrared light sensors arranged inside the toilet to receive the infrared light irradiated by the light source, and
the server device comprises:
a processor configured to inspect a health condition of the subject based on inspection data acquired based on a strength of the infrared light received by the infrared light sensors, and output a result of the inspection.
